(19) United States
(12) Patent Application Publication
(10) Pub. No.: US 2022/0238211 A1
(43) Pub. Date: Jul. 28, 2022

(54) Notification System and Notification Method

(71) Applicant: Nippon Telegraph and Telephone Corporation, Tokyo (JP)

(72) Inventors: Kei Kuwabara, Tokyo (JP); Toshishige Shimamura, Tokyo (JP); Ken Sakaguchi, Tokyo (JP); Hitoshi Okikawa, Tokyo (JP); Shingo Uchiyama, Tokyo (JP); Kensuke Kurihashi, Tokyo (JP)

(21) Appl. No.: 17/617,092

(22) PCT Filed: May 28, 2020
(86) PCT No.: PCT/JP2020/021076

§ 371 Date: Dec. 7, 2021

(30) Foreign Application Priority Data
Jun. 12, 2019 (JP) 2019-109431

(51) Int. Cl. G16H 40/20; G16H 40/67; A61B 5/364; A61B 5/361; A61B 5/00

(52) U.S. Cl. CPC G16H 40/20; G16H 40/67; A61B 5/364; A61B 5/6804; A61B 5/7282; A61B 5/7235; A61B 5/361

(57) ABSTRACT

A wearable device includes: a biological information acquisition device that acquires biological information of a subject; a biological information storage device that stores biological information; an abnormality determination device that determines whether or not there is an abnormality and an abnormality level in the biological information of the subject; a notification destination determination device that, if an abnormality has occurred in the biological information of the subject, acquires notification destination information that corresponds to the abnormality level that was determined by the abnormality determination device; a notification destination database in which notification destination information for use when an abnormality has occurred in the biological information of the subject is stored for each abnormality level in advance; and a notification device that transmits information that includes the abnormality level and the determination result of the abnormality determination device to a notification destination device indicated by the information.

### CROSS-REFERENCE TO RELATED APPLICATIONS

This application is a national phase entry of PCT Application No. PCT/JP2020/021076, filed on May 28, 2020, which claims priority to Japanese Application No. 2019-109431, filed on Jun. 12, 2019, which applications are hereby incorporated herein by reference.

### TECHNICAL FIELD

The present invention relates to a notification system and a notification method for acquiring biological information of a subject using a wearable device, and giving an external notification regarding an abnormality in the biological information.

### BACKGROUND

There are multiple types of electrocardiographs and applications thereof, but long-term electrocardiography is particularly effective in the detection of sudden electrocardiographic abnormalities such as paroxysmal atrial fibrillation. Recent years have seen the development of electrocardiographs and electrodes that allow a subject to perform electrocardiogram measurement in their daily life (see PTL 1 to 3). With such electrocardiographs, long-term electrocardiographic measurement becomes possible, and countermeasures against a sudden life crisis are being actively discussed.

However, in the systems disclosed in PTL 1 to 3, a medical institution is directly notified when a subject has an abnormality, and therefore if many abnormality notifications for many subjects are received, there is a possibility that the medical institution cannot respond to all of the abnormality notifications.

### CITATION LIST

### Patent Literature

PTL 1 Japanese Patent Application Publication No. 2009-201877

PTL 2 Japanese Patent Application Publication No. 2009-189570

PTL 3 Japanese Patent Application Publication No. 2014-54391

### SUMMARY OF THE INVENTION

### Technical Problem

Embodiments of the present invention have been made to solve the foregoing problems, and an object of embodiments of the present invention is to provide a notification system and a notification method capable of reducing the possibility that a medical institution cannot respond to an abnormality that has occurred in a subject.

### Means for Solving the Problem

A notification system according to an aspect of embodiments of the present invention includes: a biological information acquisition unit configured to acquire biological information of a subject; an abnormality determination unit configured to determine whether or not there is an abnormality in the biological information of the subject, and determine an abnormality level if it was determined that there is an abnormality; a storage unit in which notification destination information for use when an abnormality has occurred in the biological information of the subject is stored for each abnormality level in advance; a notification destination determination unit configured to, if an abnormality occurred in the biological information of the subject, reference the storage unit and acquire notification destination information that corresponds to the abnormality level determined by the abnormality determination unit; and a notification unit configured to transmit, to a notification destination device indicated by the information acquired by the notification destination determination unit, information that includes the abnormality level and indicates a determination result of the abnormality determination unit.

Also, in a configuration example of the notification system of embodiments of the present invention, the biological information acquisition unit, the abnormality determination unit, the storage unit, the notification destination determination unit, and the notification unit are provided in a wearable device worn by the subject.

In another configuration example of the notification system of embodiments of the present invention, the biological information acquisition unit is provided in a wearable device worn by the subject, the abnormality determination unit, the storage unit, the notification destination determination unit, and the notification unit are provided in a notification device on a network, the wearable device further includes a biological information transmission unit configured to transmit the biological information of the subject to the notification device, and the notification device further includes a biological information reception unit configured to receive the biological information of the subject that was transmitted by the wearable device.

In another configuration example of the notification system of embodiments of the present invention, the biological information acquisition unit, the storage unit, the notification destination determination unit, and the notification unit are provided in a wearable device worn by the subject, the abnormality determination unit is provided in a notification device on a network, the wearable device further includes a biological information transmission unit configured to transmit the biological information of the subject to the notification device, and a determination result reception unit configured to receive information that includes the abnormality level and indicates a determination result of the abnormality determination unit from the notification device, and the notification device further includes a biological information reception unit configured to receive the biological information of the subject that was transmitted by the wearable device, and a determination result transmission unit configured to transmit information that includes the abnormality level and indicates a determination result of the abnormality determination unit to the wearable device.

In another configuration example of the notification system of embodiments of the present invention, the biological information transmission unit transmits the biological information of the subject to the notification device if transmission permission has been received from the subject.

In another configuration example of the notification system of embodiments of the present invention, the notification device further includes a judge notification unit configured to transmit the biological information of the subject that was transmitted by the wearable device, to a device in possession of a predetermined judge or a server device that provides a web site that can be viewed by the judge, and a judge determination result reception unit configured to receive information indicating a determination result of the judge, and the abnormality determination unit determines whether or not there is an abnormality in the biological information of the subject based on the biological information of the subject that was transmitted by the wearable device and the determination result of the judge that was received by the judge determination result reception unit, and determines an abnormality level if it was determined that there is an abnormality.

In another configuration example of the notification system of embodiments of the present invention, the notification device further includes a reward calculation unit configured to, if the biological information reception unit received information that was transmitted by the wearable device, calculate a reward that is to be given to the subject who is wearing the wearable device, and a reward information transmission unit configured to transmit the reward calculated by the reward calculation unit to an external device.

Also, a notification method according to one aspect of embodiments of the present invention includes: a first step of acquiring biological information of a subject; a second step of determining whether or not there is an abnormality in the biological information of the subject; a third step of determining an abnormality level if it was determined that there is an abnormality in the biological information of the subject; a fourth step of, if an abnormality occurred in the biological information of the subject, referencing a storage unit in which notification destination information for use when an abnormality has occurred is stored for each abnormality level in advance, and acquiring notification destination information that corresponds to the abnormality level that was determined in the third step; and a fifth step of transmitting the information obtained in the second and third steps to a notification destination device indicated by the information that was acquired in the fourth step.

Effects of embodiments of the Invention

According to embodiments of the present invention, it is determined whether or not there is an abnormality in the biological information of a subject, and if it was determined that there is an abnormality, the abnormality level is determined, a notification destination that corresponds to the abnormality level is selected, and information that includes the abnormality level and indicates the determination result obtained by the abnormality determination unit is transmitted to a notification destination device, and therefore it is possible to smoothly and suitably respond to the subject and reduce the possibility that a medical institution cannot respond to the occurrence of an abnormality in the subject.

### BRIEF DESCRIPTION OF THE DRAWINGS

FIG. 1 is a diagram illustrating an overview of a notification system according to a first embodiment of the present invention.

FIG. 2 is a block diagram showing a configuration of a wearable device of the notification system according to the first embodiment of the present invention.

FIG. 3 is a flowchart illustrating the operation of the wearable device of the notification system according to the first embodiment of the present invention.

FIG. 4 is a diagram showing an example of information registered in a notification destination database of the notification system according to the first embodiment of the present invention.

FIG. 5 is a block diagram showing a configuration of the wearable device of the notification system according to a second embodiment of the present invention.

FIG. 6 is a block diagram showing a configuration of the wearable device and a notification device of the notification system according to a third embodiment of the present invention.

FIG. 7 is a flowchart illustrating the operation of the wearable device of the notification system according to the third embodiment of the present invention.

FIG. 8 is a flowchart illustrating the operation of the notification device of the notification system according to the third embodiment of the present invention.

FIG. 9 is a diagram showing an example of information registered in the notification destination database of the notification system according to the third embodiment of the present invention.

FIG. 10 is a block diagram showing a configuration of the wearable device of the notification system according to a fourth embodiment of the present invention.

FIG. 11 is a flowchart illustrating the operation of the wearable device of the notification system according to the fourth embodiment of the present invention.

FIG. 12 is a diagram showing an example of information registered in the notification destination database of the notification system according to a sixth embodiment of the present invention.

FIG. 13 is a block diagram showing a configuration of the notification device of the notification system according to a seventh embodiment of the present invention.

FIG. 14 is a block diagram showing a configuration of the notification device of the notification system according to an eighth embodiment of the present invention.

FIG. 15 is a flowchart illustrating the operation of the notification device of the notification system according to the eighth embodiment of the present invention.

FIG. 16 is a diagram showing an example of judge information stored in a judge information storage unit of the notification device of the notification system according to the eighth embodiment of the present invention.

FIG. 17 is a block diagram showing a configuration of a server device of the notification system according to a ninth embodiment of the present invention.

FIG. 18 is a diagram showing an example of a web page viewed by a judge in the ninth embodiment of the present invention.

FIG. 19 is a diagram showing an example of a web page viewed by a subject in the ninth embodiment of the present invention.

FIG. 20 is a block diagram showing a configuration of the wearable device and the notification device of the notification system according to a twelfth embodiment of the present invention.

FIG. 21 is a flowchart illustrating the operation of the wearable device of the notification system according to the twelfth embodiment of the present invention.

FIG. 22 is a flowchart illustrating the operation of the notification device of the notification system according to the twelfth embodiment of the present invention.

FIG. 23 is a block diagram showing a configuration of the notification device of the notification system according to a thirteenth embodiment of the present invention.

FIG. 24 is a flowchart illustrating the operation of the notification device of the notification system according to the thirteenth embodiment of the present invention.

FIG. 25 is a flowchart illustrating abnormality level determination processing performed by an abnormality determination unit of the notification system according to a fourteenth embodiment of the present invention.

FIG. 26 is a diagram showing an example of the correspondence relationship between subject symptoms and abnormality levels.

FIG. 27 is a diagram showing an example of a band used when a subject wears the wearable device in a fifteenth embodiment of the present invention.

FIG. 28 is a block diagram showing a configuration example of a computer that realizes the wearable device and the notification device of the notification system according to any of the first to fifteenth embodiments of the present invention.

### DETAILED DESCRIPTION OF ILLUSTRATIVE EMBODIMENTS

### First Embodiment

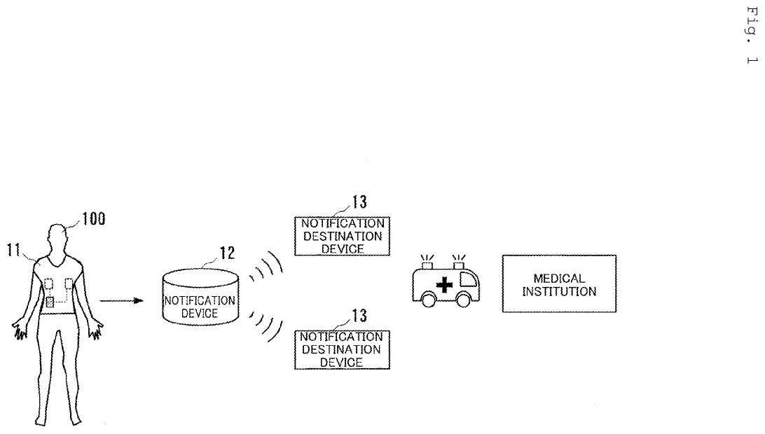

Hereinafter, an example of the present invention will be described with reference to the drawings. FIG. 1 is a diagram illustrating an overview of a notification system according to a first embodiment of the present invention.

A wearable device 11 is attached to the body of a subject 100. FIG. 1 shows an example of a shirt-type wearable device 11, but it goes without saying that the present invention is not limited to being a shirt-type wearable device.

The wearable device 11 has a wireless communication function or a wired communication function, and includes functionality for transmitting biological information of the subject 100. In the present invention, electrocardiographic data is envisioned as the biological information, but pulse data, acceleration data, respiration data, body temperature data, myoelectric data, or the like may be included. By acquiring a plurality of pieces of biological information in this way, it is possible to determine the condition of the subject from multiple viewpoints.

A notification device 12 receives the biological information of the subject from the wearable device 11 and determines whether or not there is an abnormality in the biological information of the subject. The notification device 12 includes functionality for determining the abnormality level if it has been determined that there is an abnormality in the biological information of the subject, and also for changing the notification destination device 13 that is to receive the determination result and the abnormality level according to the abnormality level.

In the present invention, the notification device 12 is provided on a network, and the wearable device 11 and the notification device 12 may be connected to each other via the network, or the notification device 12 may be incorporated in the wearable device 11. If the wearable device 11 and the notification device 12 are integrated, it is not necessary to separately provide the notification device 12, and the notification system can be constructed easily.

Examples of the notification destination device 13 include a device operated by a security company contracted by the subject or a relative or the like of the subject, a device such as a smartphone in the possession of a relative or the like of the subject, and a device such as a smartphone in the possession of the subject. The notification destination device 13 is not a device at a medical institution. The reason why a medical institution is not a notification destination is that if the system directly notifies a medical institution of the condition of the subject, the device requires medical approval as a device related to medical practice, and various complicated procedures are required before the notification system can be put into service.

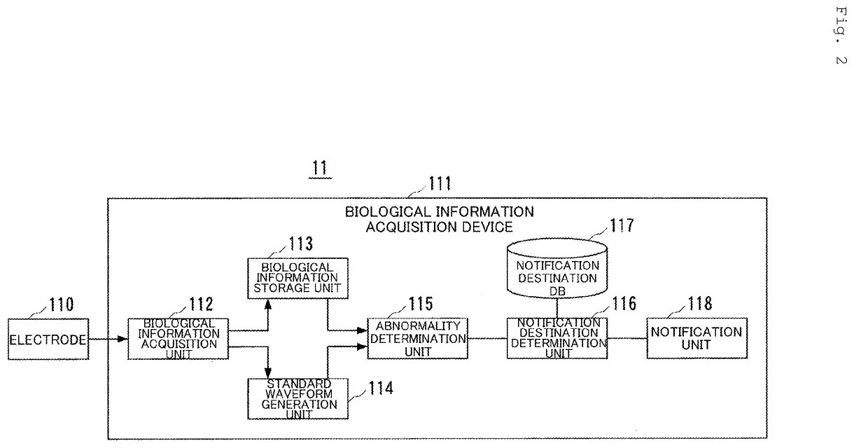

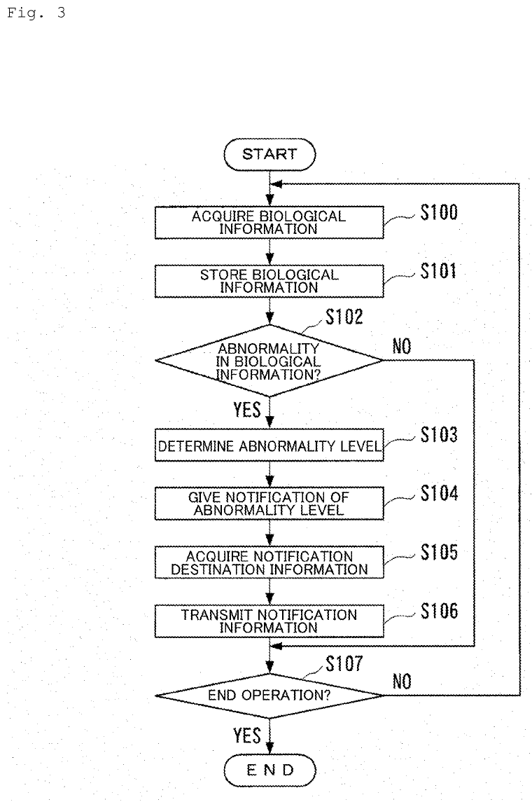

Next, the configuration and operation of the wearable device 11 will be described. FIG. 2 is a block diagram showing the configuration of the wearable device 11, and FIG. 3 is a flowchart illustrating the operation of the wearable device 11. In the present embodiment, an example is described in which the functionality of the notification device 12 is also incorporated in the wearable device 11.

As shown in FIG. 2, the wearable device 11 includes an electrode 110 and a biological information acquisition device 111. The biological information acquisition device 111 includes a biological information acquisition unit 112 that acquires biological information of the subject, a biological information storage unit 113 that stores biological information, a standard waveform generation unit 114 that generates a standard waveform of biological information, an abnormality determination unit 115, a notification destination determination unit 116, a notification destination database (hereinafter, notification destination DB) 117, and a notification unit 118. The abnormality determination unit 115 determines whether or not there is an abnormality in the biological information of the subject, and determines the abnormality level if it has been determined that there is an abnormality. When an abnormality appears in the biological information of the subject, the notification destination determination unit 116 acquires information regarding the notification destination that corresponds to the abnormality level determined by the abnormality determination unit 115. The notification destination DB 117 is a storage unit in which information regarding which notification destination is to be used when an abnormality occurs in the biological information of the subject is stored in advance for each abnormality level. The notification unit 118 transmits information that includes the abnormality level and indicates the determination result of the abnormality determination unit 115 to the notification destination device indicated by the information acquired by the notification destination determination unit 116.

The biological information acquisition unit 112 of the wearable device 11 acquires biological information (an electrocardiographic waveform) of the subject via the electrode no attached to the subject (step S100 in FIG. 3). Note that the electrode 110 of the wearable device 11 has a well-known configuration, and therefore will not be described in detail.

The biological information received by the biological information acquisition unit 112 is stored in the biological information storage unit 113 of the wearable device 11 (step S101 in FIG. 3). Note that the memory that constitutes the biological information storage unit 113 may be an external memory that can be removed from the biological information acquisition device 111, or may be a built-in memory.

The standard waveform generation unit 114 of the wearable device 11 has a function of generating a standard waveform of an electrocardiographic waveform for each subject. A standard waveform is generated for each person by superimposing multiple waveforms using a well-known method. Further, multiple general electrocardiographic waveform patterns may be prepared, and a pattern that is close to the normal electrocardiographic waveform of the subject may be selected and set in advance. Using general electrocardiographic waveform patterns in this way makes it possible to simplify the device design and reduce the cost.

The abnormality determination unit 115 of the wearable device 11 compares the electrocardiographic waveform indicated by the biological information of the subject stored in the biological information storage unit 113 with the standard waveform generated by the standard waveform generation unit 114, and determines whether or not an abnormality appears in the biological information of the subject (step S102 in FIG. 3).

As examples of methods for determining the presence or absence of an abnormality in biological information (electrocardiographic waveform), there is a method in which an abnormality such as sinus bradycardia or sinus tachycardia is detected based on the P wave interval (P-P interval) of an electrocardiographic waveform, and a method in which an abnormality such as a risk of atrial fibrillation is detected based on the fact that the R wave interval (R-R interval) of an electrocardiographic waveform is irregular and f waves are observed. Note that such abnormality determination methods that are based on an electrocardiographic waveform are well-known, and therefore will not be described in detail. Also, an abnormality may be determined based on only the biological information of the subject, or an abnormality may be determined by comparing the biological information with a standard waveform as described above.

If it was determined that there is an abnormality in the biological information of the subject (YES in step S102), the abnormality determination unit 115 determines the abnormality level of the biological information (step S103 in FIG. 3), and transmits an abnormality level flag that indicates the abnormality level to the notification destination determination unit 116 (step S104 in FIG. 3). The abnormality level may be determined from among any number of levels, but in this example, is determined from among five levels, namely 5, 4, 3, 2, and 1.

In the case of determining an abnormality based on only the biological information of the subject, a normal threshold value indicating a guideline normal value is set in advance for each determination item (e.g., P-P interval, R-R interval, heart rate, QRS width from Q wave to S wave, and P wave height). If a determination item of the biological information is within the normal range defined by the normal threshold value, the abnormality determination unit 115 determines that it is normal. If a determination item of the biological information is outside the normal range defined by the normal threshold value, the abnormality determination unit 115 determines that an abnormality has occurred. Also, the abnormality determination unit 115 may estimate a symptom of the subject (e.g., atrial fibrillation, extra systole, angina, ventricular fibrillation, or myocardial infarction) based on the determination item in which the abnormality has occurred.

Also, multiple abnormality level determination threshold values for determining the abnormality level are set for each determination item according to amounts of deviation of the determination item in the biological information from the normal threshold value (e.g., four abnormality level determination threshold values in the case of a five-step evaluation). By comparing the amount of deviation of the determination item in the biological information from the normal threshold value with the abnormality level determination threshold values, the abnormality determination unit 115 can determine the abnormality level according to the amount of deviation of the determination item from the normal threshold value.

Also, in the case of making the abnormality determination by comparing the biological information with the standard waveform, a plurality of abnormality level determination threshold values for determining the abnormality level are set according to the amount of deviation of the biological information from the standard waveform. By comparing the amount of deviation of the biological information from the standard waveform with the abnormality level determination threshold values, the abnormality determination unit 115 can determine the abnormality level according to the amount of deviation of the biological information from the standard waveform.

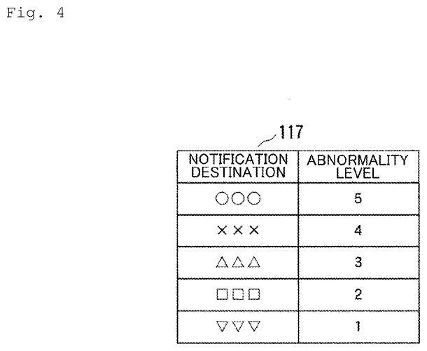

Information on a notification destination (the address of a notification destination device 13) that is to be used when an abnormality occurs in the biological information of the subject is registered in advance for each abnormality level in the notification destination DB 117 of the wearable device 11. FIG. 4 shows an example of the information registered in the notification destination DB 117. Abnormality levels and notification destination information are registered in association with each other in the notification destination DB 117.

Upon receiving the abnormality level flag from the abnormality determination unit 115, the notification destination determination unit 116 of the wearable device 11 references the notification destination DB 117 and acquires the notification destination information that corresponds to the abnormality level indicated by the abnormality level flag from the notification destination DB 117 (step S105 in FIG. 3).

The information in the notification destination DB 117 may be registered by the subject himself/herself, by a relative or the like of the subject, by a doctor at a medical institution, by a representative at a security company contracted by the subject or a relative or the like of the subject, or by a staff member of an elderly home or nursing care facility where the subject resides. Also, examples of information registration methods include a method of registration with a PC (Personal Computer) and a method of registration via a network. In the present invention, any registration method may be used.

The notification unit 118 of the wearable device 11 generates notification information that includes the determination result of the abnormality determination unit 115, and transmits the notification information to the notification destination device 13 indicated by the information acquired by the notification destination determination unit 116 (step S106 in FIG. 3). The notification information includes the determination result of the abnormality determination unit 115 (information indicating that there is an abnormality in the biological information, symptom information estimated from the determination item in which the abnormality has occurred, and the abnormality level).

The wearable device 11 performs the processing of steps S100 to S106 at regular intervals until, for example, the subject instructs the end of operation (YES in step S107 of FIG. 3).

Note that a comment regarding urgency may be added to the notification information. In this case, a comment regarding urgency may be registered in advance in the notification destination DB 117 for each abnormality level, and the notification destination determination unit 116 may be able to acquire the comment together with the notification destination information.

As described above, according to the present embodiment, when an abnormality occurs in the biological information of the subject, a notification destination device 13 is selected according to the abnormality level, and a notification of the abnormality is given to the selected notification destination device 13. Accordingly, it is possible to smoothly and suitably respond to the subject, and it is possible to reduce the possibility that a medical institution cannot respond to the occurrence of an abnormality in the subject.

When a relative or the like of the subject who is in possession of the notification destination device 13 is notified of the occurrence of an abnormality, they can take measures such as taking the subject to a medical institution or calling an ambulance.

As another example, a notification destination device 13 operated by a security company contracted by the subject may be prepared. This enables the security company to provide specialized services to subjects who have paid a contract fee.

Also, a special notification destination device 13 (a notification destination device 13 operated by a medical institution, or a notification destination device 13 related to a medical institution) may be prepared in advance for a subject who is determined to have a high degree of urgency (high abnormality level), regardless of whether or not there is a cost. As a result, it is possible to give priority to a subject who has a life-threatening risk.

Further, the notification destination device 13 may be a shared data box that can be viewed by medical institutions. Here, rather than medical institutions being directly notified of data, the shared data box is a data box in which data can only be viewed by medical institutions if they are registered.

### Second Embodiment

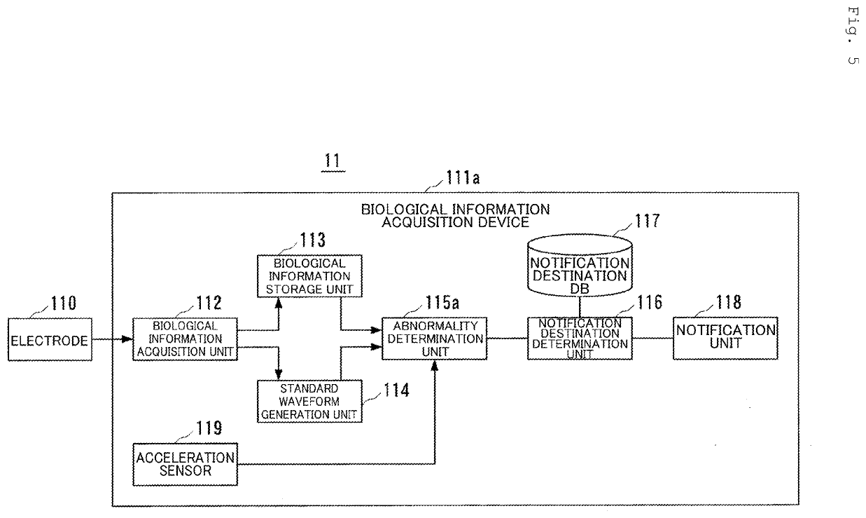

Next, a second embodiment of the present invention will be described. FIG. 5 is a block diagram showing the configuration of the wearable device 11 of the present embodiment. The wearable device 11 of the present embodiment includes the electrode 110 and a biological information acquisition device 111a.

The biological information acquisition device 111a includes the biological information acquisition unit 112, the biological information storage unit 113, the standard waveform generation unit 114, an abnormality determination unit 115a, the notification destination determination unit 116, the notification destination DB 117, the notification unit 118, and an acceleration sensor 119.

The flow of processing in the wearable device 11 is as described in FIG. 3. In the present embodiment, the abnormality determination unit 115a uses information from the acceleration sensor 119 together with the biological information of the subject stored in the biological information storage unit 113 to determine whether or not there is an abnormality in the biological information of the subject (step S102 in FIG. 3).

Because the acceleration sensor 119 can estimate the posture and movement of the subject, it is possible to make a determination based on a combination of the biological information and information on the posture and movement of the subject, such as that the subject is awake, the subject is sleeping, or the subject is walking. In this case, a normal threshold value is determined for each determination item (e.g., P-P interval, R-R interval, heart rate, QRS width from Q wave to S wave, and P wave height), and for each posture or movement of the subject.

The abnormality determination unit 115a estimates the posture or movement of the subject based on the acceleration detected by the acceleration sensor 119, and determines that an abnormality has occurred if a determination item of the biological information is outside the normal range defined by the normal threshold value that corresponds to the posture or movement of the subject. In this way, if the R-R interval is short (heart rate is high) for example, it can be determined that the subject is in a normal condition in which their heart rate is rising due to exercising, or if the heart rate is rising at rest, it can be determined that the subject is in an abnormal condition. Other aspects of the configuration are as described in the first embodiment.

### Third Embodiment

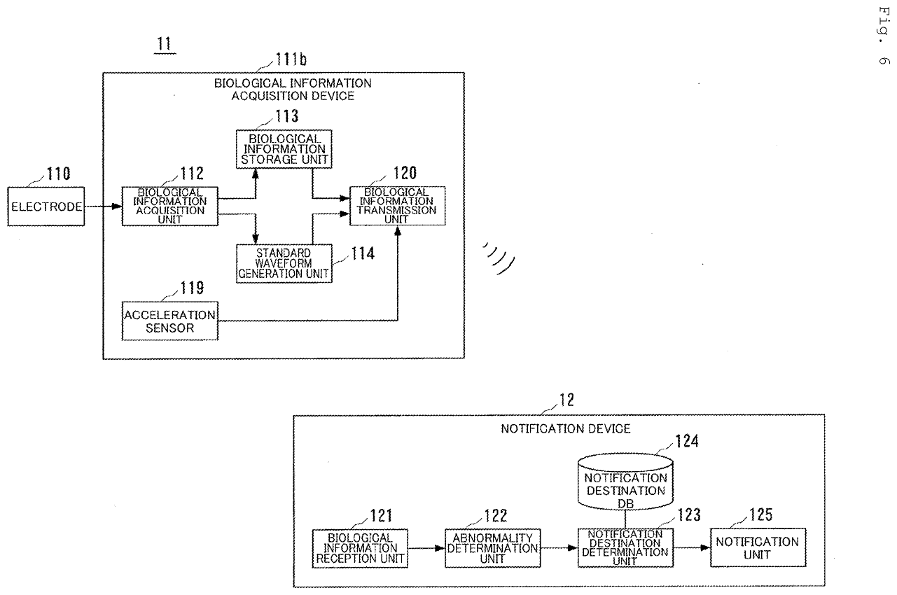

Next, a third embodiment of the present invention will be described. In the present embodiment, the case where the notification device 12 is on a network will be described. FIG. 6 is a block diagram showing the configuration of the wearable device 11 and the notification device 12 of the present embodiment. The wearable device 11 of the present embodiment includes the electrode 110 and a biological information acquisition device 111b.

The biological information acquisition device 111b includes the biological information acquisition unit 112, the biological information storage unit 113, the standard waveform generation unit 114, the acceleration sensor 119, and a biological information transmission unit 120.

The notification device 12 includes a biological information reception unit 121, an abnormality determination unit 122, a notification destination determination unit 123, a notification destination DB 124, and a notification unit 125.

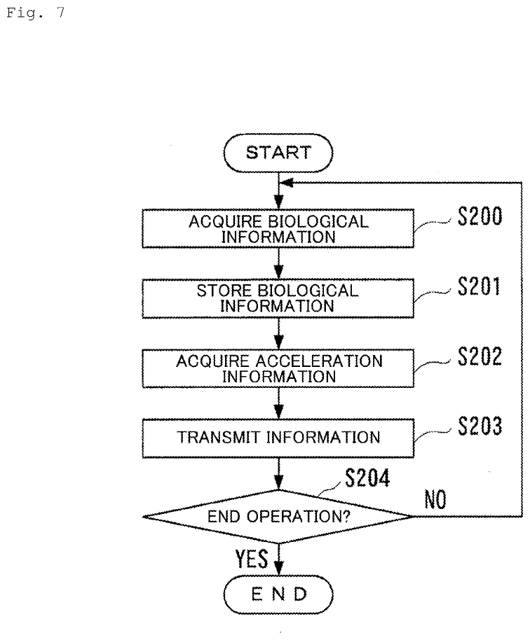

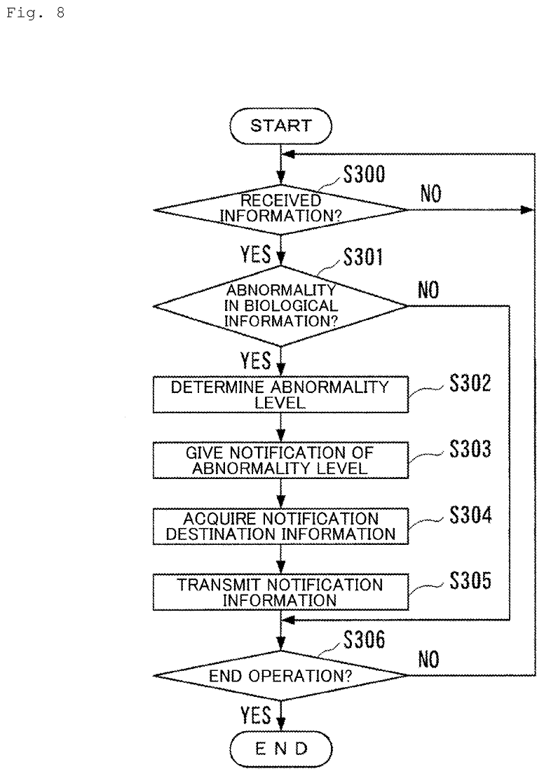

FIG. 7 is a flowchart illustrating the operation of the wearable device 11 of the present embodiment, and FIG. 8 is a flowchart illustrating the operation of the notification device 12 of the present embodiment.

The processing of steps S200 and S201 in FIG. 7 is the same as the processing of steps S100 and S101 in FIG. 3.

The biological information transmission unit 120 of the wearable device 11 acquires acceleration information detected by the acceleration sensor 119 (step S202 in FIG. 7).

The biological information transmission unit 120 transmits, to the notification device 12, a subject ID (identification information) unique to the subject, the biological information of the subject stored in the biological information storage unit 113, standard waveform information generated by the standard waveform generation unit 114, and the acceleration information detected by the acceleration sensor 119 (step S203 in FIG. 7).

The wearable device 11 performs the processing of steps S200 to S203 at regular intervals until, for example, the subject instructs the end of operation (YES in step S204 of FIG. 7).

Also, the biological information reception unit 121 of the notification device 12 receives the information transmitted by the wearable device 11 (step S300 in FIG. 8).

Similarly to the abnormality determination unit 115a, the abnormality determination unit 122 of the notification device 12 determines whether or not an abnormality has occurred in the biological information of the subject based on the biological information of the subject and the acceleration information that were transmitted by the wearable device 11 (step S301 in FIG. 8).

Then, similarly to the abnormality determination units 115 and 115a, if it was determined that there is an abnormality in the biological information of the subject (YES in step S301), the abnormality determination unit 122 determines the abnormality level of the biological information (step S302 in FIG. 8), and transmits the abnormality level flag indicating the abnormality level to the notification destination determination unit 123 (step S303 in FIG. 8). Note that the abnormality determination unit 122 may estimate a symptom of the subject based on the determination item in which the abnormality has occurred, similarly to the abnormality determination units 115 and 115a.

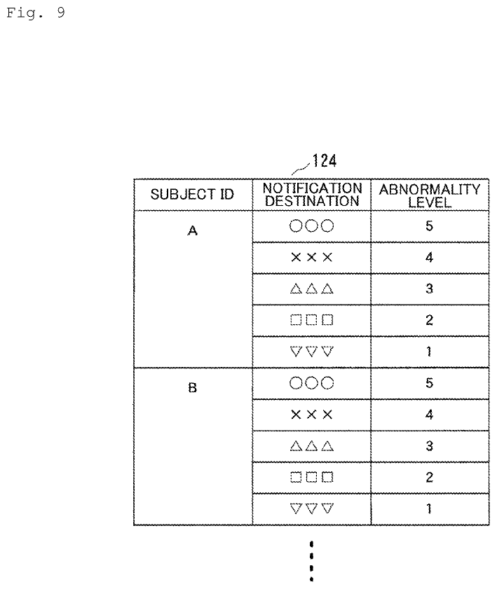

Information on a notification destination (the address of a notification destination device 13) that is to be used when an abnormality occurs in the biological information of the subject is registered in advance for each subject ID and abnormality level in the notification destination DB 124 of the notification device 12. FIG. 9 shows an example of the information registered in the notification destination DB 124. Subject IDs, abnormality levels, and notification destination information are registered in association with each other in the notification destination DB 124.

Upon receiving the abnormality level flag from the abnormality determination unit 122, the notification destination determination unit 123 of the notification device 12 references the notification destination DB 124 and acquires, from the notification destination DB 124, the notification destination information that corresponds to the subject ID and the abnormality level indicated by the abnormality level flag, which were transmitted by the wearable device 11 (step S304 in FIG. 8).

Similarly to the notification unit 118, the notification unit 125 of the notification device 12 generates notification information and transmits the notification information to the notification destination device 13 indicated by the information acquired by the notification destination determination unit 123 (step S305 in FIG. 8).

The notification device 12 performs the processing of steps S300 to S305 at regular intervals until, for example, a manager of the notification device 12 instructs the end of operation (YES in step S306 of FIG. 8).

In this way, in the present embodiment, the abnormality determination and the transmission of a notification to the notification destination device 13 can be performed by the notification device 12 on a network.

### Fourth Embodiment

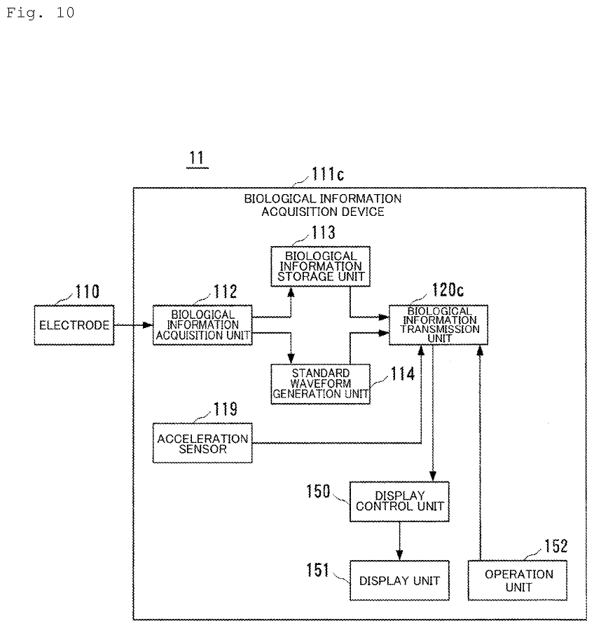

Next, a fourth embodiment of the present invention will be described. FIG. 10 is a block diagram showing the configuration of the wearable device 11 of the present embodiment. The wearable device 11 of the present embodiment includes the electrode 110 and a biological information acquisition device 111c.

The biological information acquisition device 111c includes the biological information acquisition unit 112, the biological information storage unit 113, the standard waveform generation unit 114, the acceleration sensor 119, a biological information transmission unit 120c, a display control unit 150, a display unit 151, and an operation unit 152.

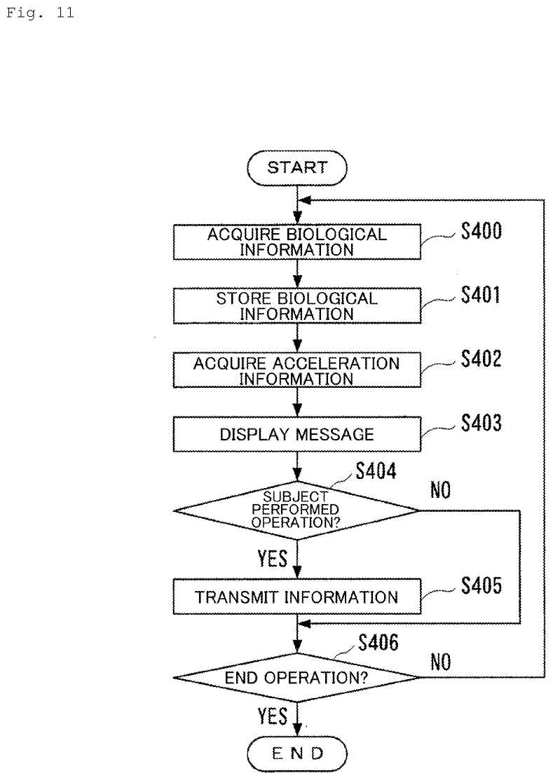

FIG. 11 is a flowchart illustrating the operation of the wearable device 11 of the present embodiment. The processing of steps S400 to S402 in FIG. 11 is the same as the processing of steps S200 to S202 in FIG. 7.

When the biological information transmission unit 120c can transmit information to the notification device 12, the display control unit 150 of the wearable device 11 of the present embodiment causes the display unit 151 to display a message asking the subject whether or not the transmission of information is permitted (step S403 in FIG. For example, the display control unit 150 causes the display unit 151 to display a message such as “Transmit information?”.

If the subject wishes for information to be transmitted to the notification device 12, the subject operates the operation unit 152 (e.g., presses a button).

If an operation was performed by the subject (YES in step S404 in FIG. the biological information transmission unit 120c of the wearable device 11 transmits, to the notification device 12, the subject ID, the biological information of the subject stored in the biological information storage unit 113, the standard waveform information generated by the standard waveform generation unit 114, and the acceleration information detected by the acceleration sensor 119 (step S405 in FIG. 11). At this time, biological information corresponding to the past several seconds, the standard waveform information, and the acceleration information may be transmitted to the notification device 12.

The configuration and operation of the notification device 12 are as described in the third embodiment.

Electrocardiographic abnormalities often have subjective symptoms that the subject is aware of. In the present embodiment, information is transmitted to the notification device 12 when the subject performs an operation on the wearable device 11, thus obtaining the effect that the notification device 12 can make a timely diagnosis of biological information that has subjective symptoms.

Note that although the case where the wearable device 11 includes the acceleration sensor 119 is described in the third and fourth embodiments, the wearable device 11 may be configured without the acceleration sensor 119. In this case, the abnormality determination unit 122 of the notification device 12 performs the processing of steps S301 and S302 similarly to the abnormality determination unit 115 of the first embodiment.

### Fifth Embodiment

In the third and fourth embodiments, an example is described in which the wearable device 11 includes the standard waveform generation unit 114, but in the third and fourth embodiments, the standard waveform generation unit 114 may be omitted. In this case, the notification device 12 may be provided with the standard waveform generation unit 114.

In the present embodiment, the amount of data transmitted by the wearable device 11 can be reduced due to providing the notification device 12 with the standard waveform generation unit 114. For this reason, the power consumption of the wearable device 11 can be reduced, and the battery life of the wearable device 11 can be extended.

Note that in the first to fifth embodiments, in the case where the abnormality determination is made based on only the biological information of the subject, or in the case where the abnormality determination is made based on the biological information of the subject and the acceleration information, the standard waveform generation unit 114 is not necessary.

Sixth Embodiment

Instead of the subject ID used in the third to fifth embodiments, the IP (Internet Protocol) address of the wearable device 11 or the ID of the SIM (Subscriber Identity Module) card for communication of the wearable device 11 may be used as the ID for identifying the subject.

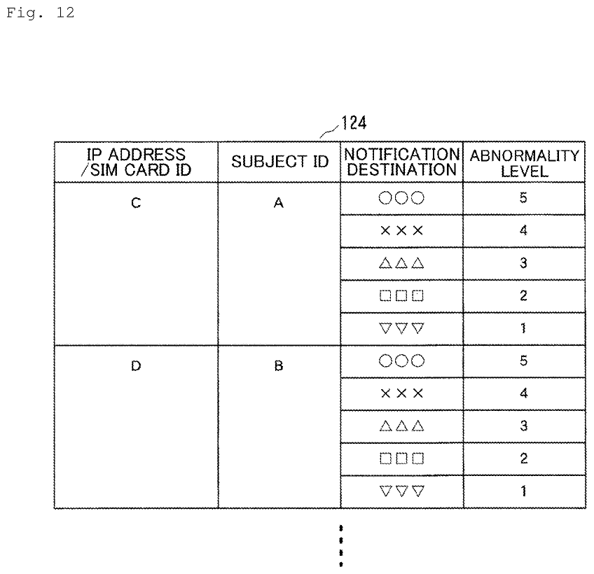

FIG. 12 shows an example of the information registered in the notification destination DB 124 of the notification device 12 of the present embodiment. An IP address or SIM card ID of the wearable device 11, a subject ID, an abnormality level, and notification destination information are registered in association with each other in the notification destination DB 124 of the present embodiment.

The biological information transmission unit 120 or 120c of the wearable device 11 transmits, to the notification device 12, the IP address or SIM card ID of the wearable device 11, the biological information of the subject stored in the biological information storage unit 113, the standard waveform information generated by the standard waveform generation unit 114, and the acceleration information detected by the acceleration sensor 119 (step S203 in FIG. 7 and step S405 in FIG. 11).

Upon receiving the abnormality level flag from the abnormality determination unit 122, the notification destination determination unit 123 of the notification device 12 references the notification destination DB 124, and acquires, from the notification destination DB 124, the notification destination information that corresponds to the IP address or SIM card ID and the abnormality level indicated by the abnormality level flag, which were transmitted by the wearable device 11 (step S304 in FIG. 8).

Other aspects of the configuration are as described in the third to fifth embodiments.

In the present embodiment, the wearable device 11 is not provided with the subject ID that can identify the subject, and therefore security can be improved.

### Seventh Embodiment

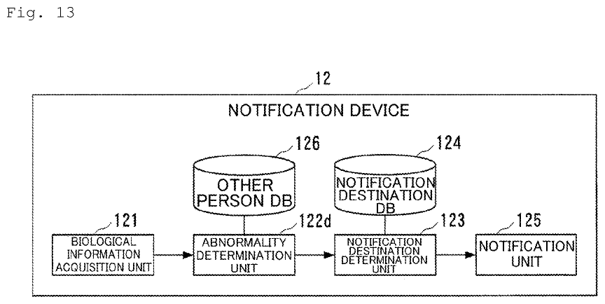

Next, a seventh embodiment of the present invention will be described. FIG. 13 is a block diagram showing the configuration of the notification device 12 of the present embodiment.

The notification device 12 of the present embodiment includes the biological information reception unit 121, an abnormality determination unit 122d, the notification destination determination unit 123, the notification destination DB 124, the notification unit 125, and an other person database (hereinafter, other person DB) 126.

The flow of processing in the notification device 12 is similar to that of the third to sixth embodiments, and therefore the operation of the notification device 12 of the present embodiment will be described with reference to FIG. 8.

The processing of steps S300 and S301 in FIG. 8 is as described in the third embodiment.

The other person DB 126 stores the electrocardiographic waveforms of people who have been determined to have an abnormality in the past and the abnormality levels thereof, or the values of determination items of the biological information of people who have been determined to have an abnormality in the past and the abnormality levels thereof.

Similarly to the abnormality determination units 115 and 115a, the abnormality determination unit 122d of the notification device 12 determines whether or not there is an abnormality in the biological information based on the biological information of a subject (or biological information and acceleration information) transmitted by the wearable device 11 of the subject (step S301 in FIG. 8).

Then, if it was determined that there is an abnormality in the biological information of the subject (YES in step S301), the abnormality determination unit 122d determines the abnormality level of the biological information (step S302 in FIG. 8). At this time, the abnormality determination unit 122d uses other person information that has been registered in advance in the other person DB 126.

Specifically, the abnormality determination unit 122d determines the abnormality level according to the amount of deviation of the electrocardiographic waveform of the subject with respect to the electrocardiographic waveform of another person registered in the other person DB 126. At this time, the abnormality determination unit 122d compares the electrocardiographic waveform of the subject with the electrocardiographic waveform of another person. If the electrocardiographic waveform of the other person and the electrocardiographic waveform of the subject substantially match each other, the abnormality determination unit 122d sets the abnormality level of the subject to the same value as the abnormality level of the other person used in the comparison. Also, if the electrocardiographic waveform of the subject deviates to the normal side with respect to the electrocardiographic waveform of the other person, the abnormality determination unit 122d sets the abnormality level of the subject to a value that is lower than the abnormality level of the other person used in the comparison. Further, if the electrocardiographic waveform of the subject deviates to the abnormal side with respect to the electrocardiographic waveform of the other person, the abnormality determination unit 122d sets the abnormality level of the subject to a value that is higher than the abnormality level of the other person used in the comparison. Note that the amount of deviation may be determined using a cross-correlation function between the electrocardiographic waveform of the subject and the electrocardiographic waveform of the other person used in the comparison.

In an alternative example, the abnormality determination unit 122d determines the abnormality level according to the amount of deviation of the value of a determination item of the subject with respect to the value of a determination item of the other person registered in the other person DB 126. If the value of the determination item of the other person and the value of the determination item of the subject substantially match each other, the abnormality determination unit 122d sets the abnormality level of the subject to the same value as the abnormality level of the other person used in the comparison; if the value of the determination item of the subject deviates to the normal side with respect to the value of the determination item of the other person, the abnormality level of the subject is set lower than the abnormality level of the other person used in the comparison; and if the value of the determination item of the subject deviates to the abnormal side of the value of the determination item of the other person, the abnormality level of the subject is set higher than the abnormality level of the other person used in the comparison.

Other aspects of the configuration are as described in the third to sixth embodiments. In the present embodiment, it is possible to compare the biological information of the subject with a very large amount of biological information of other people who have been determined to have an abnormality in the past, and the longer the system operation time is, the more accurate the abnormality determination can be.

### Eighth Embodiment

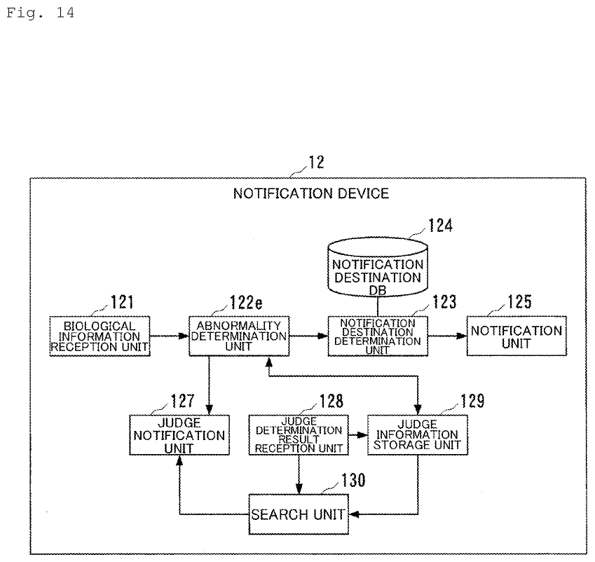

Next, an eighth embodiment of the present invention will be described. FIG. 14 is a block diagram showing the configuration of the notification device 12 of the present embodiment. The notification device 12 of the present embodiment includes the biological information reception unit 121, an abnormality determination unit 122e, the notification destination determination unit 123, the notification destination DB 124, the notification unit 125, a judge notification unit 127, a judge determination result reception unit 128, a judge information storage unit 129, and a search unit 130.

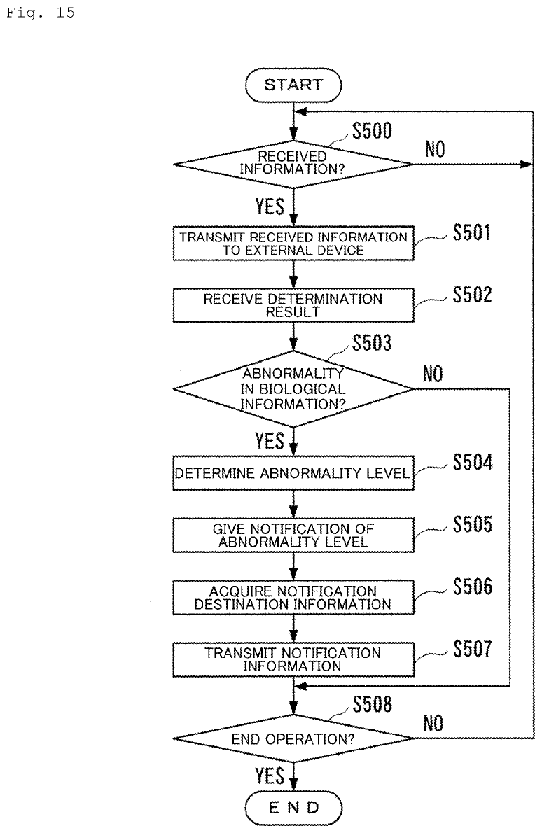

FIG. 15 is a flowchart illustrating the operation of the notification device 12 of the present embodiment. When the biological information reception unit 121 receives information transmitted by the wearable device 11 (step S500 in FIG. 15), the judge notification unit 127 of the notification device 12 transmits the received information to a predetermined external device of the judge (step S501 in FIG. 15).

The judge views the information that was transmitted by the notification device 12, determines whether or not there is an abnormality in the biological information of the subject, and in a case of determining that there is an abnormality, determines the abnormality level. One example of a judge is a doctor of a medical institution that has been contracted in advance.

The judge operates a device such as a PC that is in their possession or inputs information on a web page that will be described later and transmits the determination result (information on the presence/absence of an abnormality in the biological information, and an estimated symptom) and a judge comment to the notification device 12.

The judge determination result reception unit 128 of the notification device 12 stores the information received from the judge's device (e.g., PC), or a server device that provides the later-described web page, in the judge information storage unit 129 together with the biological information of the subject (step S502 in FIG. 15).

Similarly to the first to sixth embodiments, the abnormality determination unit 122e of the notification device 12 determines whether or not there is an abnormality in the biological information of the subject (step S503 of FIG. 15), and in the case of determining that there is an abnormality in the biological information of the subject, determines the abnormality level of the biological information (step S504 in FIG. 15). However, if there is a difference between the determination made by the abnormality determination unit 122e and the determination result of the judge, the abnormality determination unit 122e gives priority to the determination result of the judge.

The processing of steps S505 to S508 of FIG. 15 is the same as the processing of steps S303 to S306 of FIG. 8. Other aspects of the configuration are as described in the third to sixth embodiments.

As described above, in the present embodiment, a method of obtaining a judgement from a judge via a network is used as the abnormality determination method.

In the first to seventh embodiments, the notification system determines whether or not there is an abnormality in the biological information of the subject, but a determination result obtained by an expert can be added to this determination.

Information may be transmitted to the judge in step S501 by being transmitted to the judge's device at a predetermined address, or the judge notification unit 127 may transmit the information to a specific server device operated by the judge or a third party.

Also, when determining the abnormality level, the abnormality determination unit 122e determines the abnormality level based on a comment from the judge (step S504). Specifically, the abnormality determination unit 122e determines the abnormality level based on a symptom included in the comment (e.g., atrial fibrillation, extra systole, angina, ventricular fibrillation, or myocardial infarction). If there are comments from multiple judges, the comment of the judge with the highest evaluation may be adopted, or the comments of multiple judges may be weighted according to the evaluation of the judges when determining the abnormality level. A method for evaluating judges will be described later.

Also, the seventh embodiment and the eighth embodiment may be combined so that access rights for the other person DB 126 are given only to a highly evaluated judge.

The biological information stored in the other person DB 126 has high medical value, and therefore can be used to motivate the judge to judge the biological information of the subject.

### Ninth Embodiment

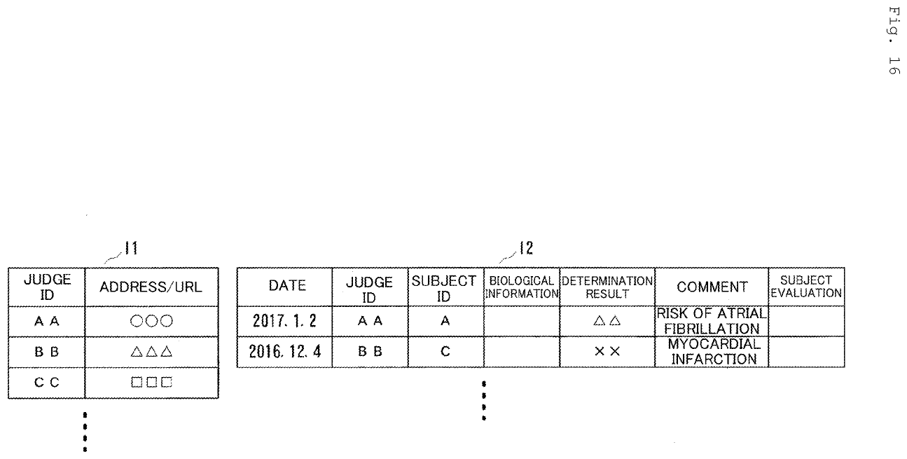

Next, a ninth embodiment of the present invention will be described. The present embodiment describes the judge information used in the eighth embodiment. FIG. 16 is a diagram showing an example of the judge information stored in the judge information storage unit 129.

Judge IDs unique to judges and the addresses of the judges' devices or the URLs (Uniform Resource Locator) of websites are registered in advance as judge information I1 in the judge information storage unit 129. Based on this information, the judge notification unit 127 of the notification device 12 transmits information to a judge's device, or transmits information to a server device operated by a judge or a third party.

Further, date information regarding when a judge determined that there is or is not an abnormality in the biological information of a subject, a judge ID, a subject ID, biological information of a subject, a determination result of a judge, a comment of the judge, and evaluation information determined by a subject for a judge are registered in association with each other as judge information I2 in the judge information storage unit 129.

The evaluation information determined by the subject for the judge serves as the status of the judge and can be used as important weighting information when determining the abnormality level. The evaluation information determined by the subject for the judge can also be used as the evaluation of the judge (doctor) or medical institution.

### Tenth Embodiment

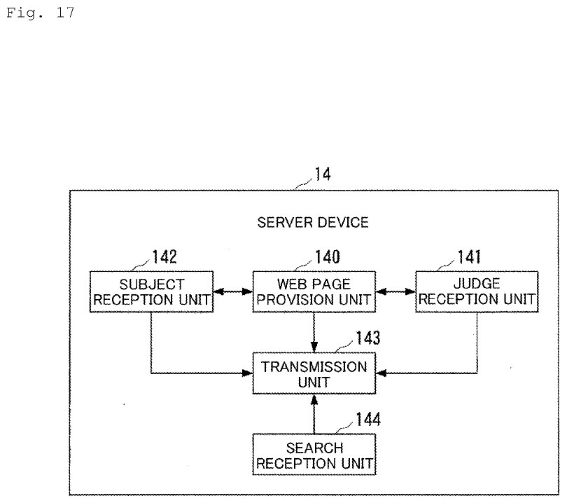

Next, a tenth embodiment of the present invention will be described. FIG. 17 is a block diagram showing the configuration of the server device of the present embodiment. The server device 14 includes a web page provision unit 140 that provides a web page for a judge site on the network, a judge reception unit 141, a subject reception unit 142, a transmission unit 143, and a search reception unit 144. The server device 14 is operated by a judge or a third party other than a judge.

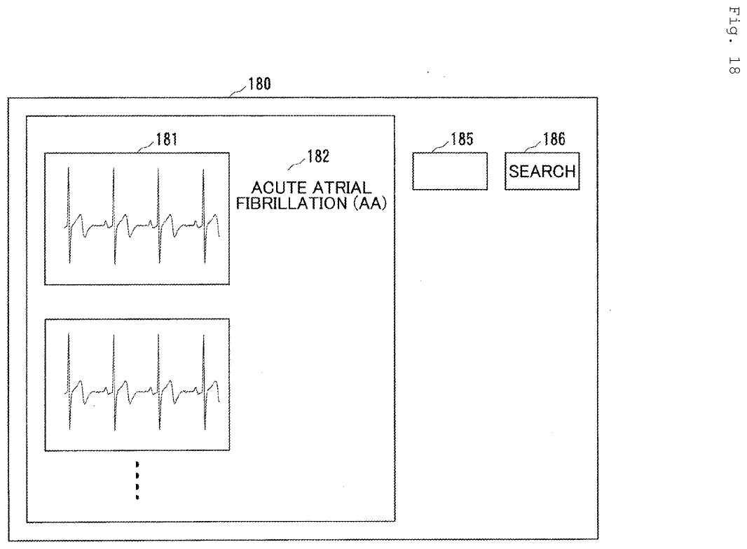

FIG. 18 is a diagram showing an example of a web page 180 viewed by a judge in the present embodiment. The web page provision unit 140 of the server device 14 publishes the web page of the judges site on the network. Access is restricted so that only the judge and the subject can view the web page of the judge. Also, among the information that can be viewed by the judge, the subject can only view information about himself/herself.

The web page provision unit 140 has an authentication function for identifying the judge and the subject. Such an authentication function is a well-known technique, and therefore will not be described in detail.

In the web page 180 in FIG. 18, 181 denotes an electrocardiographic waveform of a subject. Also, 182 denotes a comment made by a judge regarding the electrocardiographic waveform of the subject.

The judge reception unit 141 of the server device 14 displays the comment entered by the judge on the web page 180.

The transmission unit 143 of the server device 14 transmits, to the notification device 12, a judge ID, the ID of the subject to be judged, the determination result (information on the presence or absence of an abnormality in the biological information, and an estimated symptom) input by the judge via the judge reception unit 141, and the comment from the judge. Due to transmitting such information to the notification device 12 in this way, judge information 12 is stored in the judge information storage unit 129 of the notification device 12 as described above. Note that the subject ID is included in the information transmitted by the notification device 12.

In the present embodiment, the judge inputs a comment, but the judge may select a comment from among comment options prepared in advance. If the judge selects a comment from among the options, there are fewer fluctuations in comments, and data management becomes easier. Also, the time and effort required for judge input can be reduced.

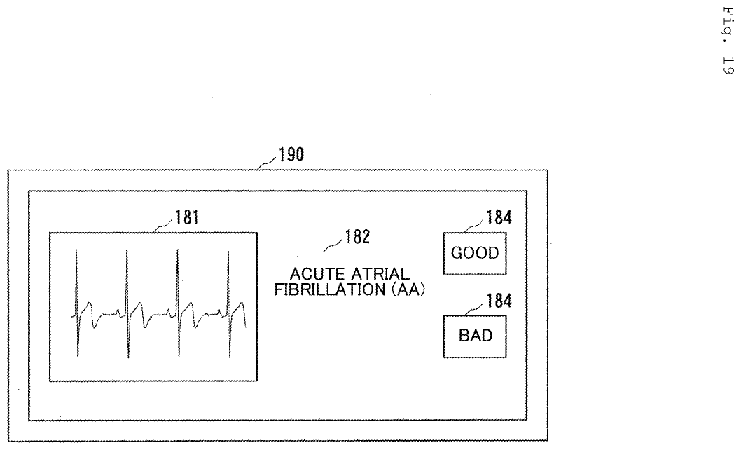

FIG. 19 is a diagram showing an example of a web page 190 viewed by the subject on a PC, a smartphone, or the like in the present embodiment. The web page provision unit 140 of the server device 14 provides the web page 190 so that among the information that can be viewed by the judge, the subject can only view information about himself/herself. As described above, the web page provision unit 140 has a subject authentication function in order to identify the subject.

Also, the web page provision unit 140 displays an evaluation button 184 for operation by the subject on the web page 190. In the example of FIG. 19, a “good” evaluation button 184 for the subject to evaluate the judge positively and a “bad” evaluation button 184 for the subject to evaluate the judge negatively are displayed.

Upon viewing the comment displayed on the web page 190, in the case of giving the judge a positive evaluation, the subject clicks the “good” evaluation button 184 on their own PC, smartphone, or the like. Also, in the case of giving the judge a negative evaluation to the judge, the subject clicks the “bad” evaluation button 184 on their own PC, smartphone, or the like.

The subject reception unit 142 of the server device 14 receives the evaluation information for the judge entered by the subject on the web page 190.

The transmission unit 143 of the server device 14 transmits, to the notification device 12, the ID of the judge who is to be evaluated, the ID of the subject who input the evaluation information, and the evaluation information that was input by the subject via the subject reception unit 142. In this way, by transmitting the evaluation information for the judge given by the subject to the notification device 12, the evaluation information is stored in the judge information storage unit 129 of the notification device 12 as described above.

Note that in the example of FIG. 19, an example of a two-stage evaluation of good/bad is shown, but evaluations may be given in multiple stages. This enables giving judges a more granular evaluation.

Further, in the example of FIG. 19, only the comment of one judge is displayed, but the comment of multiple judges may be displayed so that the subject can evaluate multiple judges.

### Eleventh Embodiment

Next, an eleventh embodiment of the present invention will be described. In the present embodiment, the configuration of the server device 14 is as described in the tenth embodiment.

As shown in FIG. 18, a keyword input field 185 and a search button 186 are displayed on the web page 180 viewed by the judge.

By operating their own PC or the like, the judge inputs a keyword in the keyword input field 185 on the web page 180, and clicks the search button 186.

The search reception unit 144 of the server device 14 receives the keyword input by the judge. The transmission unit 143 of the server device 14 transmits, to the notification device 12, the keyword that was input by the judge via the search reception unit 144.

Upon receiving the keyword from the server device 14, the search unit 130 of the notification device 12 references the judge information storage unit 129 and extracts the judge information I2 that includes the keyword.

The judge notification unit 127 of the notification device 12 then transmits the judge information I2 that was extracted by the search unit 130 to the server device 14.

The web page provision unit 140 of the server device 14 displays the judge information I2 that was transmitted by the notification device 12 on the web page 180 that is viewed by the judge.

In this way, the judge can view the biological information of other subjects for which a symptom similar to the symptom of the subject is described in the comment column, and can utilize the vast amount of information stored in the judge information storage unit 129 as medical data.

### Twelfth Embodiment

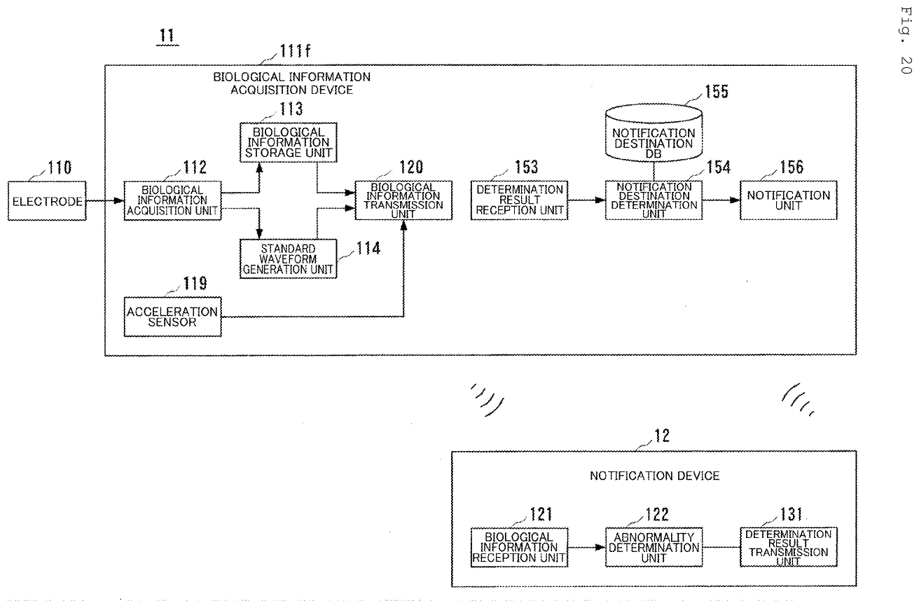

Next, a twelfth embodiment of the present invention will be described. FIG. 20 is a block diagram showing the configuration of the wearable device 11 and the notification device 12 of the present embodiment. The wearable device 11 of the present embodiment includes the electrode 110 and a biological information acquisition device 111f.

The biological information acquisition device 111f includes the biological information acquisition unit 112, the biological information storage unit 113, the standard waveform generation unit 114, the acceleration sensor 119, the biological information transmission unit 120, a determination result reception unit 153, a notification destination determination unit 154, a notification destination DB 155, and a notification unit 156.

The notification device 12 includes the biological information reception unit 121, the abnormality determination unit 122, and a determination result transmission unit 131.

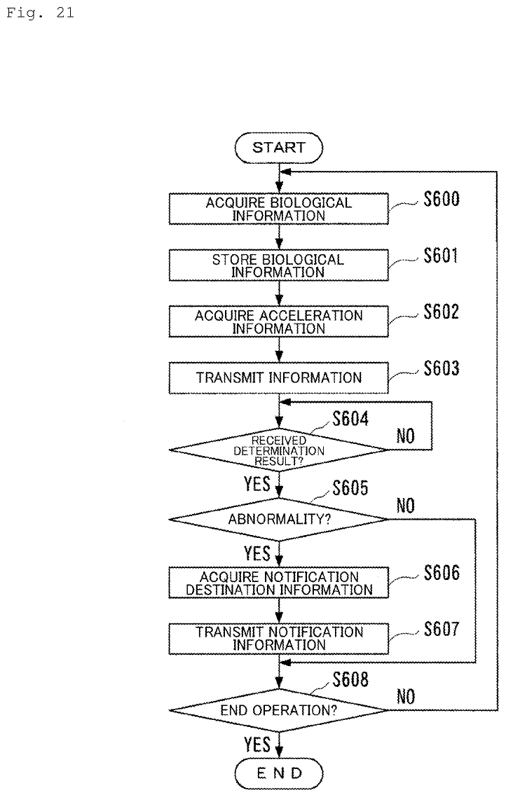

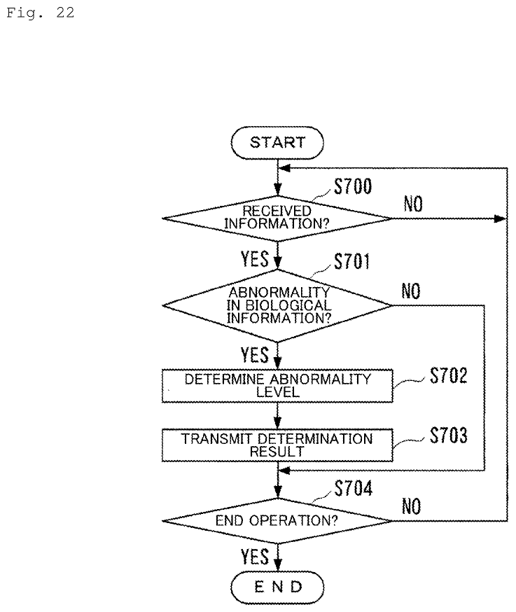

FIG. 21 is a flowchart illustrating the operation of the wearable device 11 of the present embodiment, and FIG. 22 is a flowchart illustrating the operation of the notification device 12 of the present embodiment.

The processing of steps Shoo to S603 of the wearable device 11 shown in FIG. 21 is the same as the processing of steps S200 to S203 of FIG. 7.

Also, the processing of steps S700 to S702 of the notification device 12 shown in FIG. 22 is the same as the processing of steps S300 to S302 of FIG. 8.

The determination result transmission unit 131 of the notification device 12 transmits to the wearable device 11, information on the determination result of the abnormality determination unit 122 (information indicating that there is an abnormality in the biological information, symptom information estimated from the determination item in which the abnormality has occurred, and an abnormality level) (step S703 in FIG. 22).

The notification device 12 performs the processing of steps S700 to S703 at regular intervals until, for example, the administrator of the notification device 12 instructs the end of operation (YES in step S704 of FIG. 22).

Also, if the determination result reception unit 153 receives a determination result from the notification device 12 (YES in step S604 of FIG. 21) and the determination result indicates that there is an abnormality (YES in step S605 of FIG. 21), the notification destination determination unit 154 of the wearable device 11 references the notification destination DB 155 and acquires notification destination information that corresponds to the abnormality level indicated by the abnormality level flag from the notification destination DB 155 (step S606 in FIG. 21). The configuration of the notification destination DB 155 is the same as that of the notification destination DB 117.

The notification unit 156 of the wearable device 11 generates notification information that includes the determination result received from the notification device 12, and transmits the notification information to the notification destination device 13 indicated by the information that was acquired by the notification destination determination unit 154. (step S607 in FIG. 21).

The wearable device 11 performs the processing of steps S600 to S607 at regular intervals until, for example, the subject instructs the end of operation (YES in step S608 of FIG. 21).

In the present embodiment, the abnormality determination regarding biological information is performed by the notification device 12 on the network as in the third embodiment, but the selection of the notification destination device 13 and the transmission of a notification to the notification destination device 13 are performed in the wearable device 11 as in the first and second embodiments.

Accordingly, the present embodiment has an advantage of not requiring the management of personal information on the network. In particular, notification destination information for devices that are used only for about a week, such as an Holter ECG, are more easily managed locally than on a network.

Needless to say, the present embodiment may be applied to the fourth to eleventh embodiments.

### Thirteenth Embodiment

Next, a thirteenth embodiment of the present invention will be described. In the present embodiment, an example will be described in which, in the case where abnormality determination regarding biological information is performed by the notification device 12 on a network as in the third to twelfth embodiments, the subject is prompted to transmit data on the network.

As described in the first and second embodiments, in the present invention, the wearable device 11 can have the functionality of the notification device, and therefore the subject needs to feel a certain advantage to transmit their own biological information to the notification device 12 on the network.

In the seventh embodiment, the information in the other person DB 126 can be utilized if the biological information of subjects is transmitted to the notification device 12 on the network. Further, in the eighth embodiment, if the biological information of subjects is transmitted to the notification device 12 on the network, it is possible to expect to receive a detailed determination result from the judge.

In the present embodiment, in order to give the subject further motivation, reward points, money, or the like are given to a subject who transmits their biological information to the notification device 12 on the network.

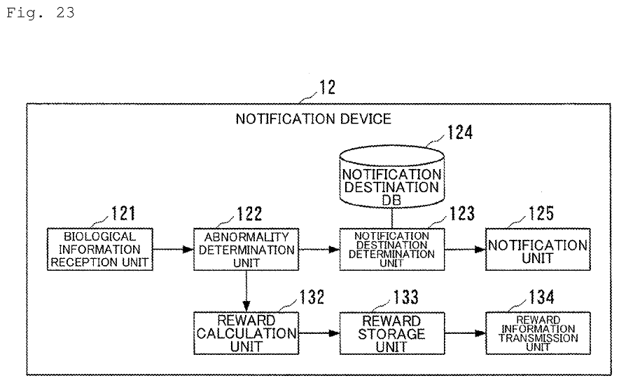

FIG. 23 is a block diagram showing the configuration of the notification device 12 of the present embodiment. The notification device 12 of the present embodiment includes the biological information reception unit 121, the abnormality determination unit 122, the notification destination determination unit 123, the notification destination DB 124, the notification unit 125, a reward calculation unit 132, a reward storage unit 133, and a reward information transmission unit 134.

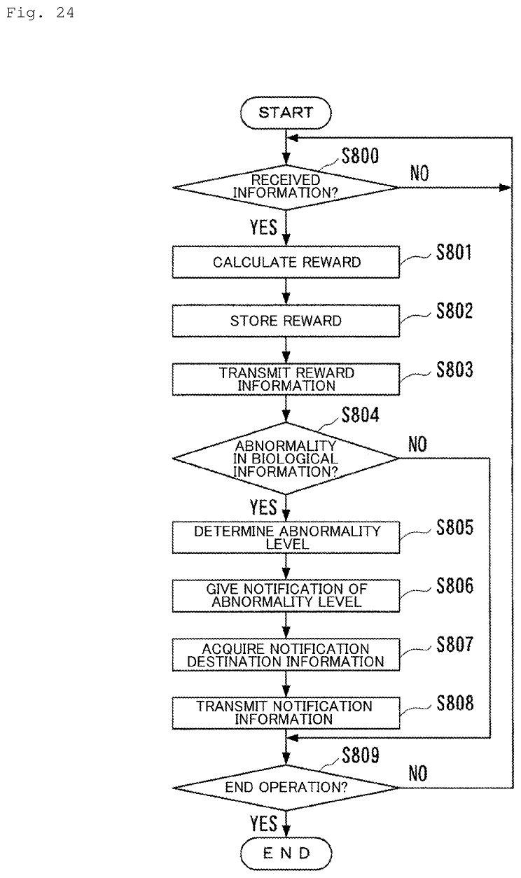

FIG. 24 is a flowchart illustrating the operation of the notification device 12 of the present embodiment. When the biological information reception unit 121 receives information transmitted by the wearable device 11 (step S800 in FIG. 24), the reward calculation unit 132 of the notification device 12 calculates a reward (reward points or money) that is to be given to the subject who is wearing the wearable device 11 (step S801 in FIG. 24). The reward calculation unit 132 may identify the subject based on the subject ID transmitted by the wearable device 11, for example, and may calculate the reward according to the number of transmissions of biological information of the subject, or may calculate the reward using another calculation method.

The value of the reward calculated by the reward calculation unit 132 is stored in the reward storage unit 133 in association with the subject ID (step S802 in FIG. 24).

The reward information transmission unit 134 of the notification device 12 transmits the subject ID and the reward value stored in the reward storage unit 133 to a predetermined external device (step 803 in FIG. 24). One example of the external device here is a server device of an insurance company contracted by the subject.

The processing of steps S804 to S809 of FIG. 24 is the same as the processing of steps S301 to S306 of FIG. 8.

In the present embodiment, it is possible to realize a framework in which information on the reward given to the subject is transmitted to the server device of the insurance company contracted by the subject for example, and thus the insurance company reduces the insurance fee by the amount of the reward. As a result, the subject can be motivated to transmit their biological information to the notification device 12 on the network.

Although the configuration of the present embodiment for giving a reward to the subject is applied to the third embodiment, it goes without saying that it may be applied to the fourth to eleventh embodiments.

Also, a configuration is possible in which the subject uploads not only current biological information but also biological information after treatment is underway. According to this configuration, the judge can evaluate the initial biological information more objectively.

### Fourteenth Embodiment

Next, a fourteenth embodiment of the present invention will be described. In the present embodiment, the operation in which the abnormality determination unit 122e determines the abnormality level of the biological information (step S504 in FIG. 15) in the eighth embodiment will be described in more detail.

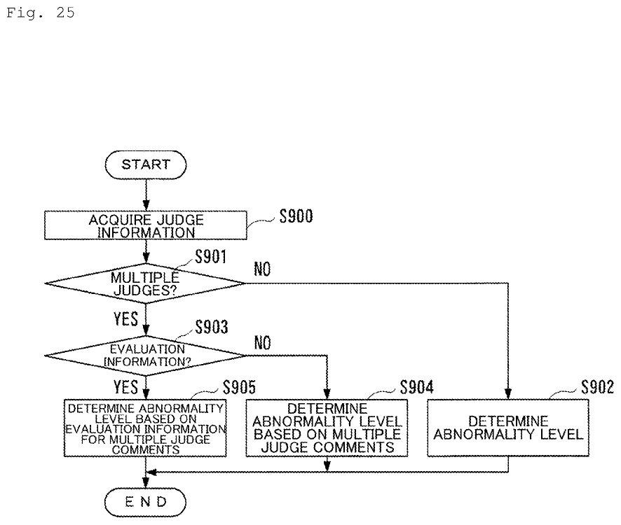

FIG. 25 is a flowchart illustrating the abnormality level determination processing of the abnormality determination unit 122e of the notification device 12. When determining the abnormality level, the abnormality determination unit 122e acquires, from the judge information storage unit 129, judge information (judge information I2 of the ninth embodiment) regarding the biological information for which the abnormality level is to be determined (step S900 in FIG. 25). Also, the abnormality determination unit 122e extracts, from the judge information (judge information I2 of the ninth embodiment), evaluation information given in the past by the subject or a person other than the subject for the judge who performed the abnormality level determination for the target biological information (step S900).

The abnormality determination unit 122e determines whether or not the acquired judge information includes comments from multiple judges (step S901 in FIG. 25).

If there are not multiple judges (NO in step S901), the abnormality determination unit 122e determines the abnormality level based on the symptom of the subject included in the comment of the judge (step S902 in FIG. 25).

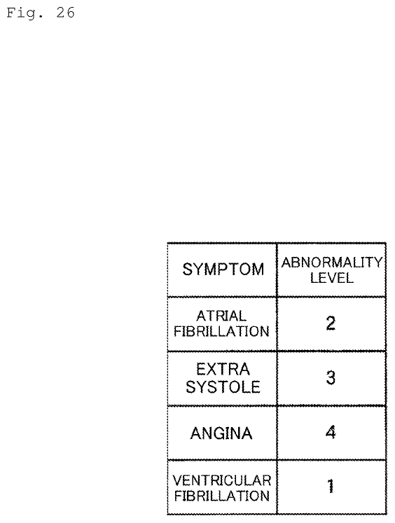

As shown in FIG. 26, the correspondence relationship between symptoms of the subject and abnormality levels has been determined in advance. In the example of FIG. 26, the higher the value of the abnormality level is, the higher the degree of urgency is. The abnormality level for atrial fibrillation is 2, the abnormality level for extrasystole is 3, the abnormality level for angina is 4, and the abnormality level for ventricular fibrillation is 1. In this way, the abnormality level can be determined based on the relationship shown in FIG. 26.

Also, if there are multiple judges (YES in step S901), the abnormality determination unit 122e determines whether or not evaluation information given to a judge in the past by the subject or a person other than the subject is included in the acquired judge information (step S903 in FIG. 25). If such evaluation information for a judge is not included (NO in step S903), the abnormality determination unit 122e determines, as the final abnormality level, the abnormality level that has the highest degree of urgency among the abnormality levels determined based on the symptoms included in the comments of the judges (step S904 in FIG. 25).

Also, if there are multiple judges, and the acquired judge information includes evaluation information for the judges (YES in step S903), the abnormality determination unit 122e determines the abnormality level based on the comments of the judges and the evaluation information (step S905 in FIG. 25).

For example, the abnormality determination unit 122e determines, as the final abnormality level, the abnormality level that was determined based on the symptom included in the comment from the judge who has the highest evaluation, from among the abnormality levels determined based on the symptoms included in the comments from the judges (step S905).

Alternatively, the abnormality determination unit 122e may weight the comments of the judges in order to determine the abnormality level (step S905). For example, assume that an evaluation score SAA for the judge AA is 5 points, an evaluation score SBB for the judge BB is 4 points, the judge AA has determined that the symptom of the subject is atrial fibrillation, and the judge BB has determined that the symptom of the subject is angina. In FIG. 26, the abnormality level for atrial fibrillation is 2, and the abnormality level for angina is 4. Accordingly, upon calculating the weight average SAA/(SAA+SBB)×2+SBB/(SAA+SBB)×4=5/(5+4)×2+4/(5+4)×4, the abnormality level is 2.9. In this way, the data of highly evaluated judges can be given more weight.

Evaluation information from the subject is not essential, but as described above, it is possible to expect an effect of being used for weighting when determining the abnormality level and improving the motivation of judges. It is also possible for a certain amount of money to be paid to a judge who has been highly evaluated by the subject. This can lead to an increase in the motivation of the judge to give a comment.

Also, judge information and employing hospital information may be managed in association with each other, and the evaluation information given by the subject for a judge may be used as an evaluation of the employing medical institution. If there are multiple judges at the same medical institution, the weight average of the evaluation information may be calculated. By evaluating medical institutions and disclosing such evaluations in this way, patients can have a basis for choosing a medical institution, and medical institutions can be motivated to become a judge and leave more comments in order to increase the number of visitors.

### Fifteenth Embodiment

Next, a sixteenth embodiment of the present invention will be described. In the present invention, it is assumed that the subject always wears the wearable device 11 when the wearable device 11 is turned on. However, the subject may temporarily remove the wearable device 11 when taking a bath or charging it. If the user forgets to turn off the power of the wearable device 11 when the wearable device 11 is removed, there is a risk that noise or the like will be detected as biological information even though the wearable device 11 cannot detect biological information.

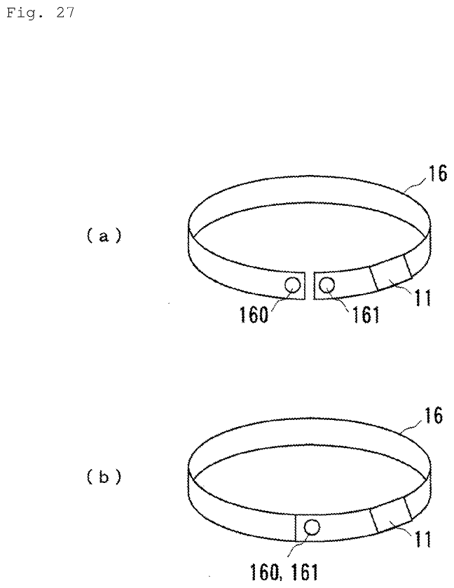

In view of this, in the present embodiment, as shown in (a) of FIG. 27 for example, electrodes (not shown) are respectively provided on snap buttons 160 and 161 of the band 16 that is fastened when the wearable device 11 is worn on the human body. When the subject wears the wearable device 11, the band 16 is tightened and the snap buttons 160 and 161 are fastened together ((b) in FIG. 27). Accordingly, the electrodes provided on the snap buttons 160 and 161 come into contact with each other, and the wearable device 11 is turned on.

Conversely, when the subject unfastens the snap buttons 160 and 161 in order to remove the wearable device the electrodes are separated from each other and the wearable device 11 is turned off.

In this way, in the present embodiment, it is possible to prevent the case where the user forgets to turn on/off the power supply when the wearable device 11 is attached/removed.

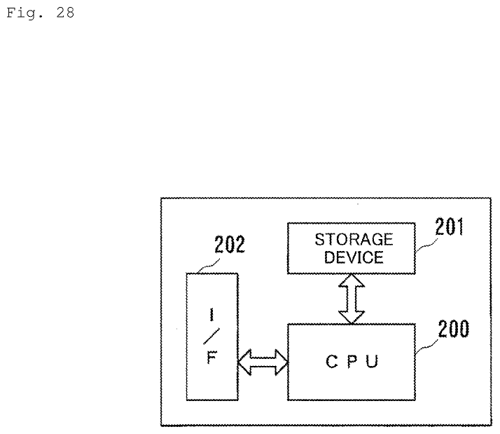

The biological information acquisition devices 111, 111a, 111b, 111c, and 111f of the wearable devices 11 described in the first to fifteenth embodiments can be realized by a computer that includes a CPU (Central Processing Unit), a storage device, and an interface, as well as a program that controls such hardware resources. A configuration example of this computer is shown in FIG. 28. The computer includes a CPU 200, a storage device 201, and an interface device (hereinafter abbreviated as I/F) 202. The I/F 202 is connected to circuits such as the biological information acquisition unit 112, the reception unit 153, the notification units 118 and 156, the acceleration sensor 119, and the biological information transmission units 120 and 120c. A program for realizing the notification method of the present invention is stored in the storage device 201 of the computer. The CPU 200 executes the processing described in any of the first to fifteenth embodiments in accordance with the program stored in the storage device 201.

Similarly, the notification device 12 can be implemented by a computer and a program. The I/F 202 of the notification device 12 is connected to circuits such as the reception units 121 and 128, the notification unit 125, the judge notification unit 127, and the transmission units 131 and 134. The CPU 200 of the notification device 12 executes the processing described in any of the first to fifteenth embodiments in accordance with the program stored in the storage device 201 of the notification device 12.

### INDUSTRIAL APPLICABILITY

Embodiments of the present invention can be applied to a technique for giving an external notification of an abnormality in the biological information of a subject.

### REFERENCE SIGNS LIST

11 Wearable device
12 Notification device
13 Notification destination device
14 Server device
110 Electrode
111, 111a, 111b, 111c, 111f Biological information acquisition device
112 Biological information acquisition unit
121, 128, 153 Reception unit
113 Biological information storage unit
114 Standard waveform generation unit
115, 115a, 122, 122c, 122d, 122e Abnormality determination unit
116, 123, 154 Notification destination determination unit
117, 124, 155 Notification destination database
118, 125, 156 Notification unit
119 Acceleration sensor
120, 120c, 131, 134, 143 Transmission unit
126 Other person database
127 Judge notification unit
129 Judge information storage unit
130 Search unit
132 Reward calculation unit
133 Reward storage unit
140 Web page provision unit
141 Judge reception unit
142 Subject reception unit
144 Search reception unit
150 Display control unit
151 Display unit
152 Operation unit.

1-8. (canceled)

9. A notification system comprising:
a biological information acquisition device configured to acquire biological information of a subject;
an abnormality determination device configured to:
determine whether or not there is an abnormality in the biological information of the subject; and
determine an abnormality level in response to determining there is the abnormality in the biological information of the subject;
a storage device configured to store notification destination information for each of one or more abnormality levels comprising the abnormality level;
a notification destination determination device configured to, in response to the abnormality in the biological information of the subject, reference the storage device and acquire first notification destination information corresponding to the abnormality level; and
a notification device configured to, in response to the abnormality in the biological information of the subject, transmit, to a notification destination device indicated by the first notification destination information, information that includes the abnormality level and indicates a determination result of the abnormality determination device.

10. The notification system according to claim 9, wherein the biological information acquisition device, the abnormality determination device, the storage device, the notification destination determination device, and the notification device are provided in a wearable device worn by the subject.

11. The notification system according to claim 9, wherein:
the biological information acquisition device is provided in a wearable device worn by the subject;
the abnormality determination device, the storage device, the notification destination determination device, and the notification device are provided in a notification device on a network;
the wearable device further includes a biological information transmission device configured to transmit the biological information of the subject to the notification device; and
the notification device further includes a biological information reception device configured to receive the biological information of the subject that was transmitted by the wearable device.

12. The notification system according to claim 11, wherein the biological information transmission device transmits the biological information of the subject to the notification device when transmission permission has been received from the subject.

13. The notification system according to claim 11, wherein:
the notification device further includes:
a judge notification device configured to transmit the biological information of the subject that was transmitted by the wearable device, to a device in possession of a predetermined judge or a server device that provides a web site that can be viewed by the predetermined judge, and
a judge determination result reception device configured to receive information indicating a determination result of the judge; and
the abnormality determination device determines whether or not there is the abnormality in the biological information of the subject based on the biological information of the subject that was transmitted by the wearable device and the determination result of the judge that was received by the judge determination result reception device.

14. The notification system according to claim 11, wherein the notification device further includes:
a reward calculation device configured to, in response to the biological information reception device receiving information that was transmitted by the wearable device, calculate a reward that is to be given to the subject; and
a reward information transmission device configured to transmit the reward calculated by the reward calculation device to an external device.

15. The notification system according to claim 9, wherein:
the biological information acquisition device, the storage device, the notification destination determination device, and the notification device are provided in a wearable device worn by the subject;
the abnormality determination device is provided in a notification device on a network;
the wearable device further includes:
a biological information transmission device configured to transmit the biological information of the subject to the notification device; and
a determination result reception device configured to receive information that includes the abnormality level and indicates a determination result of the abnormality determination device from the notification device; and
the notification device further includes:
a biological information reception device configured to receive the biological information of the subject that was transmitted by the wearable device; and
a determination result transmission device configured to transmit the information that includes the abnormality level and indicates the determination result of the abnormality determination device to the wearable device.

16. The notification system according to claim 15, wherein the biological information transmission device transmits the biological information of the subject to the notification device when transmission permission has been received from the subject.

17. The notification system according to claim 15, wherein:
the notification device further includes:
a judge notification device configured to transmit the biological information of the subject that was transmitted by the wearable device, to a device in possession of a predetermined judge or a server device that provides a web site that can be viewed by the predetermined judge, and
a judge determination result reception device configured to receive information indicating a determination result of the judge; and
the abnormality determination device determines whether or not there is the abnormality in the biological information of the subject based on the biological information of the subject that was transmitted by the wearable device and the determination result of the judge that was received by the judge determination result reception device.

18. The notification system according to claim 15, wherein the notification device further includes:
a reward calculation device configured to, in response to the biological information reception device receiving information that was transmitted by the wearable device, calculate a reward that is to be given to the subject; and
a reward information transmission device configured to transmit the reward calculated by the reward calculation device to an external device.

19. A notification method comprising:
a first step, performed by a notification system, of acquiring biological information of a subject;
a second step, performed by the notification system, of determining whether or not there is an abnormality in the biological information of the subject;
a third step, performed by the notification system, of determining an abnormality level in response to determining that there is the abnormality in the biological information of the subject;
a fourth step, performed by the notification system, of, in response to the abnormality occurring in the biological information of the subject, referencing a storage device in which notification destination information for use when an abnormality has occurred is stored for each of one or more abnormality levels in advance, and acquiring notification destination information corresponding to the abnormality level that was determined in the third step; and
a fifth step, performed by the notification system, of in response to the abnormality occurring in the biological information of the subject, transmitting information obtained in the second step and third steps to a notification destination device indicated by the notification destination information acquired in the fourth step.

20. The notification method according to claim 19, wherein the first step, the second step, the third step, the fourth step, and the fifth step are each performed by a wearable device worn by the subject, and wherein the wearable device comprises the storage device.

21. The notification method according to claim 19, wherein:
the first step is performed by a wearable device worn by the subject;
the second step, the third step, the fourth step, and the fifth step are each performed by a notification device on a network, wherein the notification device comprises the storage device;
the wearable device further includes a biological information transmission device configured to transmit the biological information of the subject to the notification device; and
the notification device further includes a biological information reception device configured to receive the biological information of the subject that was transmitted by the wearable device.
